(19) United States
(12) Patent Application Publication
(10) Pub. No.: US 2015/0327797 A1
(43) Pub. Date: Nov. 19, 2015

(54) AUDIOMETRIC SELF-TESTING

(71) Applicant: PHONAK AG, Stäfa (CH)

(72) Inventors: Nicola Schmitt, Winterthur (CH); Michael Boretzki, Ruti (CH); Harald Krueger, Affoltern am Albis (CH)

(21) Appl. No.: 14/651,738

(22) PCT Filed: Dec. 12, 2012
(86) PCT No.: PCT/EP2012/075265

§ 371 Date: Jun. 12, 2015

(51) Int. Cl. A61B 5/12; A61B 5/00

(52) U.S. Cl. CPC A61B 5/123; A61B 5/742

(57) ABSTRACT

A method includes:
a first test including adjustment by the patient of the sound pressure level of an audio stimulus to a specified hearing perception level and selection of the corresponding sound pressure level, thereby generating a first test result corresponding to the value of the sound pressure level selected by the patient;
a second test including adjustment by the patient of the sound pressure level of the audio stimulus to the same specified hearing perception level and selection of the corresponding sound pressure level, thereby generating a second test result corresponding to the value of the adjusted sound pressure level selected by the patient;
comparing the first test result and the second test result.

The result of the test is accepted or rejected based on the closeness of the test results, and feedback is provided to the patient based on this closeness.

### FIELD OF THE INVENTION

The present invention relates to measuring the hearing response of a patient. Such measurements are typically taken to determine hearing response and hearing loss to identify candidates for hearing aids, and to fit a hearing aid to a particular patient's hearing loss. By “hearing aids” we understand conventional hearing aids which may be behind-the-ear or in-the-ear types, and also cochlear implants for directly stimulating the auditory nerve.

### BACKGROUND OF THE INVENTION

Conventionally, the hearing response of a patient is measured by an audiologist by providing a series of audio stimuli to the patient, who then responds if he or she hears the stimulus. The stimuli are typically pure tones at various loudness levels and/or at different frequencies, and may be presented together with a masking noise. This is a labour-intensive process, and is subject to error from e.g. the patient being able to see the audiologist actuating the stimulus, or providing subsequent stimuli in a regular rhythm. Also, for young children the process can be difficult and intimidating.

Various methods have been proposed to overcome these problems. For instance, U.S. Pat. No. 4,862,505 proposes an audiometer enabling children to self-test their hearing by means of a computer game which rewards the child with images when the child detects the tones produced by the audiometer. Other examples of similar systems and methods are given in U.S. Pat. No. 6,925,332 and DE 190 746 722 C2.

However, such self-test methods and systems punish the patient for being hard of hearing, since the better the patient hears, the more and earlier the patient is rewarded. There thus exists an incentive for the patient to claim to hear a sound when in fact he or she does not, rendering test results less accurate. Furthermore, the patient must concentrate the whole time, which can be stressful and can lead to inaccurate results.

An object of the present invention is to overcome at least one of the above-mentioned disadvantages of the prior art.

### SUMMARY OF THE INVENTION

The object of the invention is attained by a method for measuring the hearing response of the patient, which comprises presenting at least one audio stimulus to the patient. A first test is carried out, this test comprising the patient him/herself adjusting the sound pressure level of the audio stimulus to a specified hearing perception level, such as the hearing threshold (“just audible”), a comfortable loudness level, or an uncomfortable loudness level. The method is thus a self-administered hearing test. Once the patient has adjusted the sound pressure level of the audio stimulus to the specified hearing perception level, he/she selects this sound pressure level, generating a first test result corresponding to the value of the sound pressure level selected. Subsequently, the test is repeated as a second test, again comprising adjustment by the patient of the sound pressure level of the same at least one audio stimulus to the same specified hearing perception level, which the patient then selects, generating a second test result corresponding to the value of the adjusted sound pressure level selected by the patient in the second test. The first test result and the second test result are then compared, and if they are the same within a predefined tolerance such as 10 dB or 5 dB, or even 4 dB or 2 dB, the test results are accepted as accurate and feedback is automatically provided to the patient, whereas if the test results are not the same within a predefined tolerance, again such as 10 dB or 5 dB, or even 4 dB or 2 dB, the test results are rejected as inaccurate and again feedback is automatically provided to the patient, i.e. without human intervention. Since the feedback is provided to the patient based on the similarity of a test and a retest, i.e. the first test and the second test, rather than based on the patient either hearing the audio stimulus or not, the test is less susceptible to dissimulation, since feedback is provided based on repeatability of the tests rather than the absolute hearing ability of the patient. In consequence, a harder-of-hearing patient is not disadvantaged over a better-hearing patient in terms of feedback provided, which is the case in a conventional absolute testing method.

In an embodiment of the method, the audio stimulus is at least one of a pure tone of variable loudness, a pure tone of variable frequency, and a pure tone of variable loudness embedded in noise, narrow-band noises, warble tones or natural sounds such as bird sounds. In practice, these audio stimuli have been shown to give excellent results.

In an embodiment of the method, the at least one audio stimulus comprises a plurality of audio stimuli, a first test and a second test being effected for each of the plurality of audio stimuli. This enables the method to be used for testing the hearing response of the patient over a range of frequencies, thus providing hearing response data usable for drawing up an audiogram and/or fitting a hearing aid.

In an embodiment of the method, the patient is rewarded based on the comparison of the first test result and the second test result. This reward may be in the form of animations, videos, music, free downloads or similar. The reward may be proportional to the closeness of the results of the first test and the second test, i.e. for instance if the first test result and the second test result are within e.g. 5 dB of each other, a short animation may be played, whereas if they are within e.g. 1 dB of each other, a longer animation may be played.

In an embodiment of the method, the specified hearing perception level is at least one of the hearing threshold (i.e. “barely audible”), a comfortable loudness level, and an uncomfortable loudness level. Hearing threshold gives the best results, since it is less subjective to the patient, however advantageously the patient may carry out a first test and the second test for two or more of these specified hearing perception levels to give a more complete analysis of the patient's hearing response.

In an embodiment of the method, the first test in the second test are carried out by the patient fading in and out of the audio stimulus, i.e. increasing and subsequently decreasing the loudness of the audio stimulus. The patient identifies the specified hearing perception level once as the patient fades in the audio stimulus commencing from a sound pressure level below that likely to be perceived by the patient as the specified hearing perception level, i.e. a sound pressure level so low that a normal hearing person is unlikely to perceive it, which comprises carrying out of the first test. Subsequently, the patient identifies the specified hearing perception level a second time as the patient fades out the audio stimulus commencing from a sound pressure level above that likely to be perceived by the patient as the specified hearing perception level, i.e. a sound pressure level so high that even a person with significant hearing loss can perceive it, which comprises carrying out the second test. This reduces repetition for the patient, since the two tests can be presented as a single exercise of fading in and then out the audio stimulus. It should be noted that in both cases, the patient may fade in and out the audio stimulus so as to “bracket” the specified hearing perception level.

In an embodiment of the method, the patient is not given any visual indication of the sound pressure level of the audio stimulus. This helps to prevent dissimulation of the method, since the possibility of the patient faking the results of the tests is vanishingly small since no other indication of the absolute value of the sound pressure level of the audio stimulus is provided to the patient.

In an embodiment of the method, the method is implemented as a computer game, making carrying out of the method fun for the patient, which is particularly advantageous in the case of determining the hearing response of children.

In an embodiment of the method, the predefined tolerance is between 5dB and 10 dB, or between 2dB and 4 dB. Naturally, any particular predefined tolerance can be chosen, however 5-10 dB is an effective choice for screening potential hearing aid candidates, and 2-4 dB may produce more accurate results more suited to fitting a hearing device such as a hearing aid or a cochlear implant. An object of the invention is likewise attained by a method of generating an audiogram for a patient comprising measuring the hearing response the patient according to one of the above-mentioned methods, the method being repeated as many times as necessary utilising an audio stimulus at a different frequency for each repeat of the method.

Furthermore, an object of the invention is attained by computer program product adapted to carry out any of the above-mentioned methods. This computer program product may be a computer game. Correspondingly, an object of the invention is attained by a computer readable medium comprising program code, which when executed by data processor is adapted to execute any of the above-mentioned methods.

An object of the invention is likewise attained by a system for measuring the hearing response of a patient. The system comprises an audio stimulus generator for generating at least one audio stimulus, and a manual controller in operational connection with the audio stimulus generator. The manual controller is adapted to control and to select the sound pressure level of the audio stimulus in response to input from the patient. The system comprises means adapted to carry out a first test, the means being in operative connection with the manual controller and the audio stimulus generator. The first test comprises adjustment by the patient of the sound pressure level of the at least one audio stimulus to a specified hearing perception level, such as the hearing threshold (“barely audible”), a comfortable hearing level, or an uncomfortable hearing level and selection of this sound pressure level, thereby generating a first test result corresponding to the value of the sound pressure level selected by the patient. The system further comprises means adapted to carry out a second test, these means likewise being in operative connection with the manual controller in the audio stimulus generator. The second test likewise comprises adjustment by the patient of the sound pressure level of the at least one audio stimulus to the same specified hearing perception level, such as the hearing threshold (“barely audible”), a comfortable hearing level, or an uncomfortable hearing level and selection of this sound pressure level, thereby generating a second test result corresponding to the value of the sound pressure level selected by the patient. A comparing unit is provided in operative connection with the means for carrying out a first test and with the means for carrying out a second test. The comparing unit is adapted to compare results of the first test with the results of the second test, and thereby to determine whether the result of the first test and the result of the second test are the same within a predefined tolerance or not. A patient feedback unit is provided in operative connection with the comparing unit, and is adapted to provide feedback to the patient based on output of the comparing unit. This system enables a patient to self-test his or her hearing response. Since the feedback is provided to the patient based on the similarity of a test and a retest, i.e. the first test and the second test, rather than based on the patient either hearing the audio stimulus or not, the test is less susceptible to dissimulation, since feedback is provided based on repeatability of the tests rather than the absolute hearing ability of the patient. In consequence, a harder-of-hearing patient is not disadvantaged over a better-hearing patient in terms of feedback provided, which is the case in a conventional absolute testing method.

In an embodiment of the system, the audio stimulus generator is adapted to produce at least one of a pure tone of variable loudness, a pure tone of variable frequency, and a pure tone of variable loudness embedded in noise. In practice, these audio stimuli have been shown to give excellent results.

In an embodiment of the system, the feedback is constituted by a reward. This reward may be in the form of animations, videos, music, free downloads or similar. The reward may be proportional to the closeness of the results of first test and the second test, i.e. for instance if the first test result and the second test result are within e.g. 5 dB of each other, a short animation may be played, whereas if they are within e.g. 1 dB of each other, a longer animation may be played.

In an embodiment of the system, the audio stimulus generator is adapted to produce a plurality of audio stimuli, the system being adapted to carry out a first test in the second test for each of the plurality of audio stimuli. This enables the system to be used for testing the hearing response of the patient e.g. Over a range of frequencies, thus providing hearing response data usable for drawing up an audiogram and/or fitting a hearing aid.

In an embodiment of the system, the manual controller and the audio stimulus generator are arranged such that manipulation of the manual controller by the patient causes the audio stimulus to be faded in and out, that is to say that the sound pressure level of the audio stimulus increases and then subsequently decreases in response to the manipulation of the manual controller. This reduces repetition for the patient, since the two tests can be presented as a single exercise of fading in and then out the audio stimulus.

In an embodiment of the system, the system does not provide any visual indication of the sound pressure level of the audio stimulus. This helps to prevent dissimulation of the tests, since the possibility of the patient faking the results of the tests is vanishingly small since no other indication of the absolute value of the sound pressure level of the audio stimulus is provided to the patient.

In an embodiment of the system, the predefined tolerance is between 5 and 10 dB, or between 2 and 4 dB. Naturally, any particular predefined tolerance can be chosen, however 5-10 dB is an effective choice for screening potential hearing aid candidates, and particularly 2-4 dB may produce more accurate results more suited to fitting a hearing device such as a hearing aid or a cochlear implant.

### BRIEF DESCRIPTION OF THE DRAWINGS

The invention will now be further described with reference to the appended figures, which show:

FIG. 1: A schematic flow diagram of the principle of the invention;

FIG. 2: An illustration of a first implementation of the method of the invention in game format;

FIG. 3: An illustration of a second limitation of the method of the invention in game format;

FIG. 4: A schematic of a system implementing the invention.

### DETAILED DESCRIPTION OF THE DRAWINGS

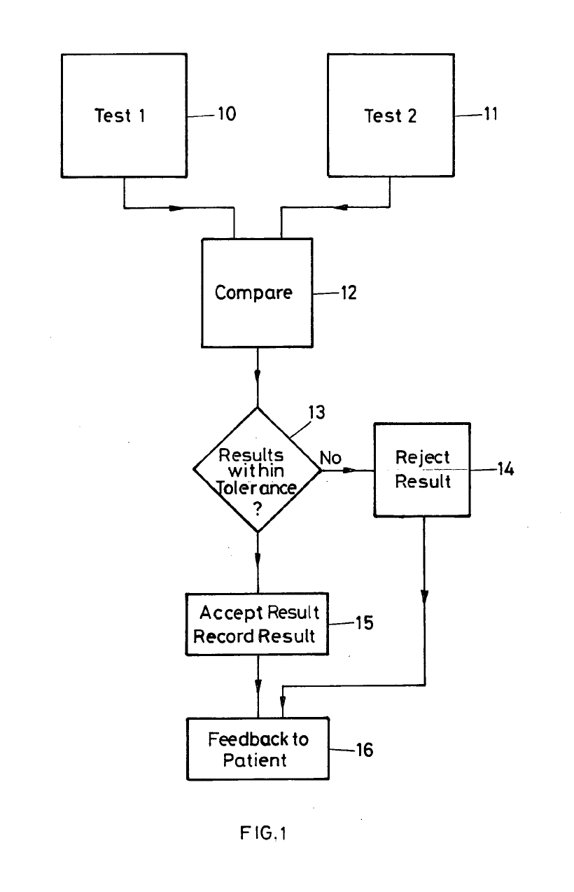

FIG. 1 illustrates schematically the principle of the invention by means of a flow diagram.

In step 10, a first test is carried out by the patient. For instance, in its simplest form the first test involves the patient adjusting a sound pressure level of an audio stimulus such as a pure tone or a pure tone presented together with a masking noise until the hearing threshold (HL), comfortable loudness level (MCL), or uncomfortable loudness level (UCL) is heard. The patient then selects this sound pressure level.

Subsequently, in step 11, a second test is carried out by the patient, in which the same audio stimulus is presented, and the same sound pressure level is adjusted and selected in the same manner.

It is advantageous in carrying out the first test and the second test that the patient does not receive any indication of absolute values being selected, since this may bias the test results.

Subsequently, in step 12 the values of the sound pressure level selected by the patient in the first test and second test are compared, and in step 13 it is determined whether the result of the comparison indicates that the values of the sound pressure levels set by the patient in each of the two tests are within a predefined tolerance, such as within 10 dB or 5 dB. If this is not the case, in step 14 the result is rejected, whereas if it is the case, the result is accepted and recorded in step 15, and in step 16 feedback is provided to the patient as to whether the comparison was within tolerance or out of tolerance.

The test may then be repeated e.g. for different frequencies, so as to build an audiogram, or to provide data for fitting a hearing aid or a cochlear implant.

As is conventional, the audio stimulus may be presented to the patient via headphones, loudspeakers in a room, a bone hearing conduction speaker, via one or more hearing aids, or via a cochlea implant.

As a non-limiting concrete example, the first test may comprise the patient adjusting the loudness (constituting the sound pressure level) of an audio stimulus of a 1 kHz tone to the hearing threshold, resulting in a sound pressure level of 55 dB. The second test then comprises the patient adjusting the same 1 kHz tone again to the hearing threshold, resulting in a sound pressure level of 58 dB. If the predefined tolerance is 5 dB, the results of the tests are accepted and recorded, and feedback is provided to the patient. The accepted and recorded test results may be averaged if required.

If, on the other hand, the results of the first test were 55 dB and the result of the second test were 65 dB and the predefined tolerance is again 5 dB, the result is rejected and feedback is given in the form of requesting that the patient repeat the first test and in the second test.

In consequence, the feedback is provided to the patient based on the similarity of the test results, rather than based on the point at which the patient hears the tone. In consequence, the feedback is not dependent on the level of hearing ability, rather on repeatability of test results. Psychologically, this makes such testing more rewarding for patients who are harder-of-hearing than is the case with feedback based on absolute values.

It is advantageous to reduce dissimulation of the tests, in the case of the result being rejected, to repeat both the first test and the second test rather than just to repeat the second test, since by repeating both tests it becomes impossible for the patient to merely keep repeating the second test by incrementing or decrementing the sound pressure level until the results of the second test is within the predefined tolerance purely by trial and error.

This trial-and-error approach can further be obstructed by using different starting sound pressure levels to avoid the patient using a systematic approach for finding the correct value.

However, since it is also possible that the patient merely makes a mistake during the first test, the result of the second test may alternatively be utilized as a first test result, and a new second test may be carried out. In consequence, the tests can be carried out until two adjacent tests give results within the predefined tolerance, the first of these tests being the “first test” and the second of these tests being the “second test”.

It is furthermore advantageous to implement the method of the invention in a game format. This adds a degree of fun to carrying out the method, and is particularly useful for measuring the hearing of children who typically enjoy games.

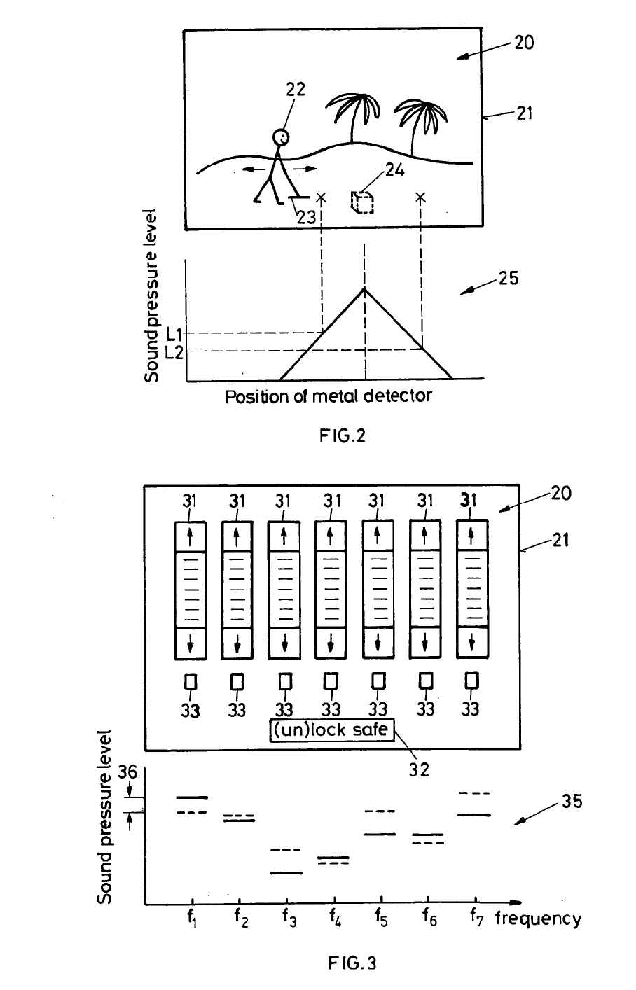

FIG. 2 illustrates a first embodiment of an implementation of the method of the invention in game format.

In this embodiment, the method of the invention is presented to the patient as a computer game 20 provided on a screen of an electronic device 21 such as a PC, a videogame console, a tablet computer or a smart phone. In this case, the game is a treasure hunting game, in which a character 22 is provided with a virtual metal detector 23. The object of the game is to identify the position of buried treasure 24, which is invisible to the patient. The patient controls the position of character 22 so as to move him to the left and to the right. An audio stimulus is presented to the patient, such as a tone of varying sound pressure. The sound pressure of the tone varies according to the position of the virtual metal detector 23, and fades in (i.e. the sound pressure level is augmented) and fades out (i.e. the sound pressure level is decremented) as the character moves across the screen 21, as illustrated schematically in graph 25. Although the sound pressure level profile in graph 25 is presented as symmetric and rising and falling linearly, this does not have to be the case, so long as the audio stimulus is faded in and then out as the character moves across the screen. Indeed, although the position of the treasure has been indicated as corresponding to the peak of the sound pressure of the audio stimulus, this also does not have to be the case, since the “treasure” is only discovered based on similarity of the first test result and the second test result, and not by identification of the sound pressure peak. It should be noted that the lowest sound pressure level should be at a level impercievable to the patient, and the peak sound pressure level should be at a level perceivable to the patient.

The first test is carried out by the patient moving the character 22 forwards or forwards and backwards in a bracketing procedure until the specified hearing level such as the hearing threshold is reached, at which point the position of the character is recorded, e.g. by clicking a mouse, pressing a key, or tapping a touchscreen, selecting (and thus saving/recording) a sound pressure level L1. The character 22 may be moved by any conventional input device such as a mouse, joystick, keyboard, trackball, touchscreen and so on. The patient continues to move the character 22 so as to carry out the second test, which comprises identifying the point at which the audio stimulus can no longer be heard, at which point again the patient selects (and thus saves/records) a sound pressure level L2. If sound pressure levels L1 and L2 are within a predefined tolerance of each other such as within 5 dB, the result is accepted, and feedback is presented to the patient, e.g. in the form of the character 22 digging up the buried treasure 24, and/or awarding of points to the patient, and/or by progressing to the next level for testing a further audio stimulus. If the sound pressure levels L1 and L2 are outside of the predefined tolerance, feedback is presented to the patient in the form of repeating the tests. Indeed, a single game “level” may incorporate a first test and a second test for determining each of the hearing threshold, comfortable loudness level, and uncomfortable loudness level, with the patient selecting each hearing level as he moves the character 22 across the screen.

The tests may then be repeated at e.g. different frequencies so as to generate a complete audiogram for the patient. Each audio stimulus frequency subject to test may be presented as a different “level” in the game, and may be distinguished for instance by different background images.

FIG. 3 illustrates a second embodiment of an implementation of the testing method of the invention in game format.

As in FIG. 2, the implementation is presented as a computer game 20 on a screen 21 of an electronic device. In this case, the method is carried out by means of a “safe cracking” game. The “safe” comprises a plurality of “locks” 31 each having e.g. an adjustment wheel and two pushbutton icons for “turning” the adjustment wheel, each of which is associated with a particular audio stimulus. Each of the “locks” 31 may be associated with a tone of a different frequency, for instance the standard frequencies making up a conventional audiogram. Selecting the up and down arrows for each of the “locks” 31 adjusts the sound pressure level associated with that particular “lock”. To “lock” the safe, the patient adjusts each of the “locks” 31 to the desired hearing level, such as hearing threshold. This corresponds to carrying out the first test for each audio stimulus associated with each “lock” 31. Once completed, the patient confirms completion of the task by selecting button 32. An animation e.g. of the “locks” being randomly changed indicates that readjustment is now necessary for unlocking. Subsequently, to “unlock” the safe, the patient readjusts each of the “locks” 31 to the same hearing level, which corresponds to carrying out the second test for each audio stimulus associated with each “lock” 31. The patient then re-confirms completion of the task by selecting button 32. Feedback is provided to the patient for instance by colouring boxes 33 green or red, each box 33 being associated with the “lock” 31 situated directly above it. If the sound pressure level selected by the patient for each individual “lock” 31 is within a predefined acceptable tolerance, such as 5 dB, the corresponding box 33 will be coloured green, and if not, the corresponding box 33 will be coloured red, and the patient must then repeat both the “locking” and “unlocking” for the “locks” 31 in question. The locking and unlocking may also be repeated several times, for example once for the left ear, once for the right ear and once, if necessary, for the repetitions.

Once all of the “locks” 31 have been successfully “locked” and then “unlocked”, the patient may be rewarded for instance by being awarded points, music and/or videos and/or animations being played, by being given access to free downloads, or by being presented with a real item such as a teddy bear represented as a virtual item inside the virtual “safe”, e.g. a digital photograph of a real teddy bear stored in the system. The sound pressure level values selected by the patient are then stored and can be used for generating audiogram, or fitting a hearing aid.

FIG. 3 further represents a graph 35 illustrating the principle of the method of the invention. In this graph, each “lock” 31 is associated with a different audio stimulus frequency, f₁-f₇. For each frequency, a solid line represents the sound pressure level selected by the patient on “locking” the corresponding “lock” 31, i.e. the sound pressure level selected by the patient in each first test, and the dashed line represents the sound pressure level selected by the patient on “unlocking” the corresponding “lock” 31, i.e. the sound pressure level selected by the patient in each second test. The acceptable threshold is illustrated at 36, and would correspond to e.g. 5 dB. From this graph it is visible that the patient has successfully “unlocked” the “locks” with frequencies f₁, f₂, f₄ and f₆, and must repeat the method for the “locks” with frequencies f₃, f₅ and f₇, since the results of these frequencies fall outside the acceptable predefined sound pressure level tolerance.

This embodiment of an implementation of the method is particularly resistant to dissimulation, since each “lock” cannot be adjusted correctly without hearing the sounds at all.

In all of the embodiments, it has been shown in practice that adjusting the audio stimulus to the hearing threshold, i.e. such that the stimulus is barely audible, gives the most reliable results, since higher hearing levels such as “middle loud”, comfortable loudness level, uncomfortable loudness level and so on are more difficult for the patient to objectively distinguish

The above described game-based implementations of the method of the invention may be provided running on a PC, a videogame console, on a tablet computer, on a smart phone as an applet in a web browser, or as an app on a smart phone or tablet computer. The software to implement the games may be provided as a computer program product on a data storage medium such as a hard drive or thumb drive, or on a web server or in an App Store.

These games may feature a high score list for comparison with other players, support for two or more people simultaneously playing and competing, whether locally or via the Internet, different game scenarios within the same audiometry, and awarding points for the closeness of the first test result and the second test result, and/or for speed of completing tests. Since the method does not penalize the hard-of-hearing over normally-hearing persons, a patient can play against a normally-hearing person such as a carer or a parent, with equal chances for either to win.

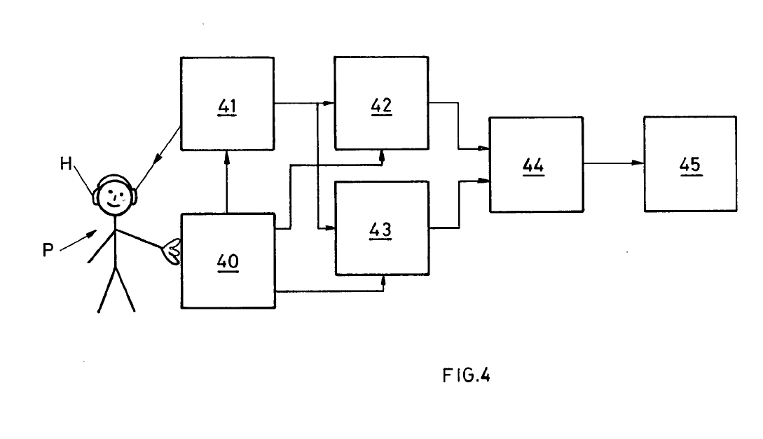

FIG. 4 illustrates the system architecture of a system according to the invention, which is adapted to carry out the method of the invention.

A manual control 40 for allowing patient P to control and select the sound pressure level of an audio stimulus generated by audio stimulus generator 41 is in operative connection with the audio stimulus generator 41 and with the manual controller 40. This audio stimulus may be for instance a pure tone of variable loudness, a pure tone of variable frequency, or a pure tone embedded in noise, and is provided to the patient P via an audio transducer H e.g. via headphones H, with which audio stimulus generator 41 is in operative connection. Alternatively, the audio stimulus may be provided via room loudspeakers, a bone conduction speaker, a cochlear implant, or one or more hearing aids in situ. In operative connection with the audio stimulus generator 41 are means for carrying out a first test 42, and means for carrying out a second test 43. Naturally, means 42 and means 43 are only schematically illustrated as being separate means, and may thus be constituted by the same means. Each test 42, 43 comprises adjustment by the patient of the sound pressure level of the at least one audio stimulus to a specified hearing perception level and selection by the patient of the resulting sound pressure level thereby generating respectively a first test result and a second test result, corresponding respectively to the value of the sound pressure level selected by the patient in the first test and the second test.

In operative connection with the means 42 and the means 43 is a comparing unit 44, adapted to compare the result of the first test with that of the second test, and thereby to determine whether the first test results and the second test result are the same within a predetermined tolerance such as within 5 dB. An output of the comparing unit 44 is in operative connection with a patient feedback unit 45 for generating feedback to the patient in response to whether the result of the first test and the result of the second test are the same within a predetermined tolerance, such as 5 dB, or are outside said predetermined tolerance. The feedback may be in proportion to the closeness of the results of the first test in the second test, for instance by providing more attractive animations or sounds based on how close the test results are.

This system may be implemented in software as a computer program product stored on a computer readable medium. The computer program in question may be a computer game, in which case the selection of the sound pressure level is implemented as part of the computer game.

The manual control 40 may be a joystick, a mouse with or without a scroll wheel, a touchscreen, a gesture sensor such as Microsoft Kinect, or similar.

The patient feedback unit may generate points in a game, music, images, animations, videos, or provide free downloads in response to the results of the first test and the second test being within the predefined tolerance, and likewise the feedback unit may generate similar music, images, animations, videos etc in response to the results of the first test of the second test being outside of the predefined tolerance, and/or may simply provide feedback in the form of making the patient repeat the first test in the second test.

For both the above-mentioned methods and systems, the audio stimulus may be calibrated as is conventional via the use of calibrated equipment, such as Aurical by GN Otometrics, calibration with a normal hearing person, or by presenting the stimulus in noise.

The invention is suitable to be carried out at an ear, nose and throat (ENT) doctor's surgery, at an audiologist's office, at home, or in a publicly accessible place such as a shopping mall.

Although the invention has been described in terms of specific embodiments, these are not to be construed as limiting the scope of the invention, which is defined solely by the scope of the appended claims.

What is claimed is:

1. Method for measuring the hearing response of a patient (P) comprising the steps of:
presenting at least one audio stimulus to the patient (P);
carrying out a first test comprising adjustment by the patient (P) of the sound pressure level of the at least one audio stimulus to a specified hearing perception level and selection of the corresponding sound pressure level, thereby generating a first test result corresponding to the value of the sound pressure level selected by the patient (P);
carrying out a second test comprising adjustment by the patient (P) of the sound pressure level of the at least one audio stimulus to the same specified hearing perception level and selection of the corresponding sound pressure level, thereby generating a second test result corresponding to the value of the adjusted sound pressure level selected by the patient (P);
comparing the first test result and the second test result;
if the first test result is the same as the second test result within a predefined tolerance, accepting the test results as accurate and automatically providing feedback to the patient (P);
if the first test result is not the same as the second test result within said predefined tolerance, rejecting the test results as inaccurate and automatically providing feedback to the patient (P).

2. Method of claim 1, wherein the audio stimulus is at least one of a pure tone of variable loudness, a pure tone of variable frequency, a pure tone of variable loudness embedded in noise, narrow-band noises, warble tones or natural sounds such as bird sounds.

3. Method of claim 1, wherein the at least one audio stimulus comprises a plurality of audio stimuli, and wherein a first test and a second test are effected for each of the plurality of audio stimuli.

4. Method according to claim 1, wherein the patient is rewarded based on the comparison of the first test result and the second test result.

5. Method of claim 4, wherein said reward is provided in proportion to the closeness of the results of the first test and the second test.

6. Method according to claim 1, wherein the specified hearing perception level is at least one of: hearing threshold, comfortable loudness level, uncomfortable loudness level.

7. Method according to claim 1, wherein the first test and the second test are carried out by the patient fading in and out the audio stimulus, the first test being effected by commencing from a sound pressure level below that likely to be perceived by the patient as the specified hearing perception level, and the second test being effected by commencing from a sound pressure level above that likely to be perceived by the patient as the specified hearing perception level.

8. Method according to claim 1, wherein the patient is not given any visual indication of the sound pressure level of the audio stimulus.

9. Method according to claim 1, wherein said method is implemented as a computer game.

10. Method according to claim 1, wherein said predefined tolerance is between 5 dB and 10 dB, or between 2 dB and 4 dB.

11. Method of generating an audiogram for a patient comprising measuring the hearing response of a patient according to the method of claim 1, the method being repeated with the at least one audio stimulus at a plurality of audio frequencies.

12. Computer program product adapted to carry out the method of claim 1.

13. Computer program product according to claim 12, wherein said computer program is a computer game.

14. Computer readable medium comprising program code, which when executed by a data processor is adapted to execute the method according to claim 1.

15. System for measuring the hearing response of a patient (P), comprising:
an audio stimulus generator (41) for generating at least one audio stimulus;
a manual controller (40) in operative connection with the audio stimulus generator (41), the manual controller (40) being adapted to control and to select the sound pressure level of the audio stimulus in response to input from the patient (P);
means (42) adapted to carry out a first test, said means (42) being in operative connection with the manual controller (40) and the audio stimulus generator (41), said first test comprising adjustment by the patient (P) of the sound pressure level of the at least one audio stimulus to a specified hearing perception level and selection by the patient of this sound pressure level, thereby generating a first test result corresponding to the value of the sound pressure level selected by the patient;
means (43) adapted to carry out a second test, said means (43) being in operative connection with the manual controller (40) and the audio stimulus generator (41), said second test comprising adjustment by the patient of the sound pressure level of the at least one audio stimulus to the same specified hearing perception level and selection of this sound pressure level by the patient, thereby generating a second test result corresponding to the value of the adjusted sound pressure level selected by the patient;
a comparing unit (44) in operative connection with the means (42) for carrying out a first test and with the means (43) for carrying out a second test, the comparing unit (44) being adapted to compare result of the first test with the result of the second test and thereby to determine whether the result of the first test and the result of the second test are the same within a predetermined tolerance;
a patient feedback unit (45) in operative connection with the comparing unit (44), the patient feedback unit (45) being adapted to give feedback to the patient based on the output of the comparing unit.

16. System according to claim 15, wherein the audio stimulus generator (41) is adapted to produce at least one of a pure tone of variable loudness, a pure tone of variable frequency, a pure tone of variable loudness embedded in noise.

17. System according to claim 17, wherein said feedback is constituted by a reward.

18. System according to claim 17, wherein said reward is in proportion to the closeness of the results of the first test and the second test.

19. System according to claim 15, wherein the audio stimulus generator (41) is adapted to produce a plurality of audio stimuli, and wherein the system is adapted to carry out a first test and a second test for each of the plurality of audio stimuli.

20. System according to claim 15 wherein the manual controller (40) and the audio stimulus generator (41) are arranged such that manipulation of the manual controller by the patient causes the audio stimulus to be faded in and out.

21. System according to claim 15 wherein the system does not provide any visual indication of the sound pressure level of the audio stimulus.

22. System according to claim 15, wherein said predefined tolerance is between 5 dB and 10 dB, or between 2 dB and 4 dB.
